(19) United States
(12) Patent Application Publication
(10) Pub. No.: US 2004/0247540 A1
(43) Pub. Date: Dec. 9, 2004

(54) Cosmetic and dermatological light-protective formulation with a content of water-soluble UV-filter substances and alkylnaphthalates

(75) Inventors: Jens Schulz, Schenefeld (DE); Voker Wendel, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/789,751

(22) Filed: Feb. 27, 2004

Related U.S. Application Data
(63) Continuation of application No. PCT/EP02/09567, filed on Aug. 28, 2002.

(30) Foreign Application Priority Data
Aug. 29, 2001 (DE) 101 41 478.1

(51) Int. Cl. A61K 7/42

(52) U.S. Cl. 424/59

(57) ABSTRACT

Cosmetic or dermatological formulations comprising at least one water-soluble UV-filter substance and at least one dialkyl naphthalate having the structural formula

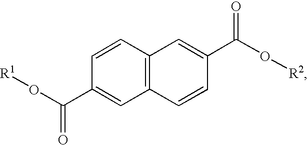

wherein R¹ and R² are independently selected from the group consisting of branched and non-branched alkyl groups with 6 to 24 carbon atoms, are useful for protecting skin from harmful effects of solar radiation, particularly from UV-A and UV-B radiation.

### CROSS-REFERENCE TO RELATED APPLICATIONS

This is a continuation application of PCT/EP02/09567, filed Aug. 28, 2002, which is incorporated herein by reference in its entirety, and also claims the benefit of German Priority Application No.101 41 478.1 filed Aug. 29, 2001.

### FILED OF THE INVENTION

The present invention relates to cosmetic and dermatological light protection preparations.

### BACKGROUND OF THE INVENTION

The harmful effect of the ultraviolet part of solar radiation on the skin is generally known. The rays have various effects on the skin as an organ depending on their particular wavelength; so-called UV-C radiation with a wavelength below 290 nm is absorbed by the ozone layer in the earth's atmosphere and therefore has no physiological significance. By contrast, rays in the range between 290 nm and 320 nm, the so-called UV-B range, cause erythema, simple sunburn or even more or less severe burns. The narrower range around 308 nm is stated to be a maximum for the erythema activity of sunlight.

Numerous compounds are known for protecting against UV-B radiation, examples thereof being derivatives of 3-benzylidenecamphor, of 4-aminobenzoic acid, of cinnamic acid, of salicylic acid, of benzophenone, and of triazine.

It has long been incorrectly assumed that the long-wavelength UV-A radiation with a wavelength between 320 nm and 400 nm has only a negligible biological effect. However, it has now been proved by many studies that UV-A radiation is far more hazardous than UV-B radiation in relation to the initiation of photodynamic, specifically phototoxic, reactions and chronic changes in the skin. It is also possible for the harmful effect of UV-B radiation to be enhanced by UV-A radiation.

Thus, it has been proved, inter alia, that even UV-A radiation under entirely normal everyday conditions is sufficient to damage within a short time the collagen and elastin fibers which are of essential importance for the structure and firmness of the skin. This results in chronic light-induced skin changes—the skin “ages” prematurely. The clinical appearance of skin aged by light includes, for example, creases and wrinkles and an irregular, furrowed relief. In addition, the areas affected by light-induced skin aging may have irregular pigmentation. The formation of brown spots, keratoses and even carcinomas or malignant melanomas is also possible. Skin aged prematurely by everyday exposure to UV is additionally distinguished by a lower activity of the Langerhans cells and a slight chronic inflammation.

About 90% of the ultraviolet radiation which reaches the earth consists of UV-A rays. Whereas UV-B radiation varies greatly depending on a large number of factors (for example season and time of day or latitude), UV-A radiation remains relatively constant from day to day irrespective of seasonal and diurnal or geographic factors. At the same time, most of the UV-A radiation penetrates into living epidermis, while about 70% of UV-B rays are retained by the stratum corneum.

It is therefore of fundamental importance that cosmetic and dermatological light protection preparations provide adequate protection both against UV-B and against UV-A radiation.

In general, the light absorption characteristics of light protection filter substances are very well known and documented, especially since most industrialized countries have positive lists for the use of such substances, which impose very strict standards on the documentation.

The use concentration of known light protection filter substances present as solids is, however, frequently limited in particular in combination with other substances to be dissolved. There are thus certain technical difficulties in formulating in order to achieve higher sun protection factors and effective UV-A protection.

Examples of advantageous UV-A filter substances are dibenzoylmethane derivatives, especially 4-(tert-butyl)-4′-methoxydibenzoylmethane (CAS No. 70356-09-1), which is sold by Givaudan with the trademark Parsole® 1789 and by Merck with the proprietary name Eusolex® 9020.

The main disadvantage of all dibenzoylmethane derivatives which absorb in the UV region is a certain instability toward UV radiation, so that these components are decomposed under the influence of UV to inactive products and are no longer available for UV absorption. Prior art preparations with a content of these substances therefore also expediently contain certain UV stabilizers such as, for example, ethylhexyl 2-cyano-3,3-diphenylacrylate(octocrylene) or 4-methylbenzylidenecamphor.

Sunburn and light erythema are the acute manifestations of the action of light. Besides the effects of UV rays which have already been described, the after-reaction of the skin further leads to a reduced sebum production and drying of the skin. To alleviate and care for these phenomena, prior art products used after sunbathing normally contain specific active ingredients such as, for example,

refatting agents and humectants,

inflammation-relieving and cooling substances,

local anesthetic substances and/or

disinfectant substances in order to prevent possible skin infections.

The so-called aftersun or aprés-soleil products are intended to cool the skin after sunbathing and to improve its ability to retain moisture, with the provision of the cooling effect playing a central role. However, there are no prior art products which protect the skin from drying and care for it adequately during UV irradiation.

### SUMMARY OF THE INVENTION

It was an object of the present invention to eliminate the prior art disadvantages and obtain in a simple manner preparations which are distinguished by very effective UV, in particular UV-A, protection and in which it is possible to dispense with the use of conventional UV stabilizers.

It was surprising and could not have been predicted by the skilled worker that the disadvantages of the prior art are remedied by cosmetic or dermatological preparations which are effective for light protection, characterized in that they comprise

(a) at least one water-soluble UV filter substance and

(b) at least one dialkyl naphthalate having the structural formula

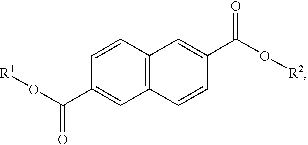

in which R¹ and R₂ are selected independently of one another from the group of branched and unbranched alkyl groups having 6 to 24 carbon atoms.

The preparations for the purposes of the present invention may, besides one or more oil phases, preferably additionally comprise one or more aqueous phases and be, for example, in the form of W/O, O/W, W/O/W or O/W/O emulsions. Such formulations may preferably also be a microemulsion, a solid emulsions (i.e. an emulsion which is stabilized by solids, e.g. a Pickering emulsion), a sprayable emulsion or a hydrodispersion.

The preparations of the invention are products which are extremely satisfactory in every respect and which are not limited to a restricted choice of raw materials. They are accordingly very particularly suitable for use as basis for preparation forms with diverse application purposes. The preparations of the invention show very good sensory and cosmetic properties such as, for example, the spreadability on the skin or the ability to penetrate into the skin, and are further characterized by very good light protection efficiency with, at the same time, excellent skin care data.

The invention therefore also relates to cosmetic or dermatological preparations which are effective for light protection, characterized in that they comprise synergistic combinations of substances from

(a) at least one water-soluble UV filter substance and

(b) at least one dialkyl naphthalate having the structural formula

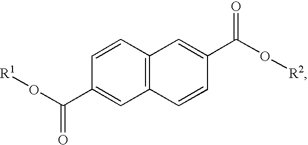

in which R¹ and R² are selected independently of one another from the group of branched and unbranched alkyl groups having 6 to 24 carbon atoms, where the effectiveness of UV protection, in particular the effectiveness of UV-A protection, of these preparations is greater than that of identical preparations which contain no substances specified in (b).

### DETAILED DESCRIPTION OF THE PREFERRED EMBODIMENTS

The effectiveness of UV protection by sunscreen compositions or of the UV filters on which they are based is usually determined in biological activity tests under standardized conditions. “Effectiveness of UV protection” means for the purposes of the present invention both the effectiveness of protection from UV-A radiation and from UV-B radiation.

A measure of the effectiveness of UV protection is represented for the purposes of the present invention for example by the sun protection factor (SPF) or else IPD values and the like.

The sun protection factor (SPF) indicates the increased time of exposure to the sun's rays made possible through use of the sunscreen composition. It is the ratio of the erythema threshold time with sunscreen composition to erythema threshold time without sunscreen composition.

The effectiveness of UV-A protection is normally tested by using the IPD method (IPD≡immediate pigment darkening). This entails—similar to the determination of the sun protection factor—measurement of a value which indicates how much longer skin protected with the light protection composition can be exposed to UV-A radiation until the same pigmentation occurs as with the unprotected skin.

Another test method which has become established throughout Europe is the Australian standard AS/NZS 2604:1997. This entails measurement of the absorption of the preparation in the UV-A region. To comply with the standard, the preparation must absorb at least 90% of the UV-A radiation in the range from 320 to 360 nm.

The invention also relates to the use of one or more dialkyl naphthalates having the structural formula

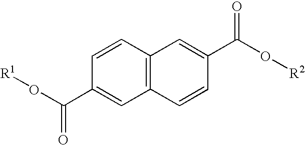

in which R¹ and R² are selected independently of one another from the group of branched and unbranched alkyl groups having 6 to 24 carbon atoms, for stabilizing dibenzoylmethane derivatives against decomposition induced by UV radiation in cosmetic or dermatological preparations which comprise at least one water-soluble UV filter substance.

It was particularly surprising that it is possible to dispense entirely with the use of further UV stabilizers, especially with the use of ethylhexyl 2-cyano-3,3-diphenylacrylate(octocrylene) or 4-methylbenzylidenecamphor, in the use according to the present invention.

Advantageous water-soluble UV filter substances for the purposes of the present invention are sulfonated UV filters, in particular:

phenylene-1,4-bis(2-benzimidazyl)-3,3′-5,5′-tetrasulfonic acid, which has the following structure:

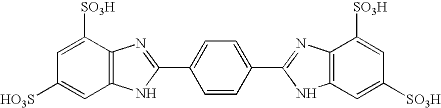

and its salts, especially the corresponding sodium, potassium or triethanolammonium salts, in particular phenylene-1,4-bis(2-benzimidazyl)-3,3′-5,5′-tetrasulfonic acid bissodium salt

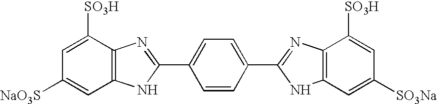

with the INCI name disodium phenyl dibenzimidazole tetrasulfonate (CAS No.: 180898-37-7), which is obtainable for example under the proprietary name Neo Heliopan A P from Haarmann & Reimer.

Further advantageous sulfonated UV filters for the purposes of the present invention are the salts of 2-phenylbenzimidazole-5-sulfonic acid, such as its sodium, potassium or its triethanolammonium salts, and the sulfonic acid itself

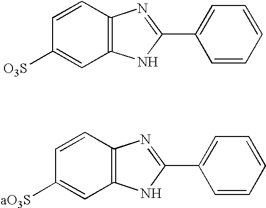

with the INCI name phenylbenzimidazole sulfonic acid (CAS No. 27503-81-7), which is obtainable for example under the proprietary name Eusolex 232 from Merck or under Neo Heliopan Hydro from Haarmann & Reimer.

Further advantageous water-soluble UV-B and/or broad-band filter substances for the purposes of the present invention are, for example, sulfonic acid derivatives of 3-benzylidenecamphor, such as, for example, 4-(2-oxo-3-bornylidenemethyl)benzene-sulfonic acid, 2-methyl-5-(2-oxo-3-bornylidenemethyl)sulfonic acid and the salts thereof.

The total amount of one or more water-soluble UV filter substances in the finished cosmetic or dermatological preparations is advantageously chosen from the range 0.01% byweightto 20% by weight, preferably from 0.1 to 10% byweight, in each case based on the total weight of the preparations.

Dialkyl naphthalates for which R¹ and/or R² represent branched alkyl groups having 6 to 10 carbon atoms are advantageous for the purposes of the present invention. Very particularly preferred for the purposes of the present invention is diethylhexyl naphthalate, which is obtainable for example under the proprietary name Hallbrite TQ™ from CP Hall or Corapan TQ™ from H&R.

It is advantageous according to the invention for cosmetic or dermatological preparations to contain from 0.001 to 30% by weight, preferably 0.01 to 20% by weight, very particularly preferably 0.5 to 15% by weight, of one or more dialkyl naphthalates.

The cosmetic or dermatological light-protective formulations of the invention may have conventional compositions and be used for cosmetic or dermatological protection from light, also for the treatment, care and cleansing of the skin and/or hair and as a makeup product in decorative cosmetics.

According to their constitution, cosmetic or topical dermatological compositions can be used for the purposes of the present invention for example as skin protection cream, cleansing milk, day or night cream etc. It is, where appropriate, possible and advantageous to use the compositions of the invention as base for pharmaceutical formulations.

For use, the cosmetic and dermatological preparations are applied in a sufficient quantity to the skin and/or hair in the manner usual for cosmetics.

The cosmetic and dermatological preparations of the invention may contain cosmetic auxiliaries as are customarily used in such preparations, e.g. preservatives, preservative assistants, bactericides, perfumes, substances to prevent foaming, dyes, pigments which have a coloring effect, thickeners, moisturizing and/or humectant substances, fillers which improve the skin feel, fats, oils, waxes or other usual ingredients of a cosmetic or dermatological formulation such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin, which is obtainable for example under the proprietary name Glydant™ from Lonza), iodopropylbutylcarbamates (e.g. those obtainable under the proprietary names Glycacil-L, Glycacil-S from Lonza and/or Dekaben LMB from Jan Dekker), parabens (i.e. alkyl p-hydroxybenzoates, such as methyl, ethyl, propyl and/or butyl paraben), phenoxyethanol, ethanol, benzoic acid and more of the like. The preservative system normally also comprises advantageously according to the invention preservative assistants such as, for example octoxyglycerin, glycine soya etc.

Particularly advantageous preparations are also obtained where antioxidants are employed as additives or active ingredients. The preparations advantageously contain according to the invention one or more antioxidants. Favorable antioxidants which may optionally be used are all antioxidants suitable or customary for cosmetic and/or dermatological applications.

It is possible and particularly advantageous for the purposes of the present invention to employ water-soluble antioxidants such as, for example, vitamins, e.g. ascorbic acid and derivatives thereof.

Further preferred antioxidants are vitamin E and derivatives thereof, and vitamin A and derivatives thereof.

The amount of antioxidants (one or more compounds) in the preparations is preferably from 0.001 to 30% by weight, particularly preferably 0.05 to 20% by weight, in particular 0.1 to 10% by weight, based on the total weight of the preparation.

If vitamin E and/or derivatives thereof represent the antioxidant(s), it is advantageous to choose the respective concentrations thereof from the range from 0.001 to 10% by weight based on the total weight of the formulation.

If vitamin A or vitamin A derivatives, or carotenes or derivatives thereof represent the antioxidant(s), it is advantageous to choose the respective concentrations thereof from the range from 0.001 to 10% by weight based on the total weight of the formulation.

It is particularly advantageous for the cosmetic preparations according to the present invention to contain cosmetic or dermatological active ingredients, with preferred active ingredients being antioxidants able to protect the skin from oxidative stress.

Further advantageous active ingredients for the purposes of the present invention are natural active ingredients and/or derivatives thereof, such as, for example, alpha-lipoic acid, phytoene, D-biotin, coenzyme Q10, alpha-glucosylrutin, carnitine, camosine, natural and/or synthetic isoflavonoids, creatine, taurine and/or β-alanine.

Formulas of the invention containing, for example, known antiwrinkle active ingredients such as flavone glycosides (especially α-glycosylrutin), coenzyme Q10, vitamin E and/or derivatives and the like are particularly advantageously suitable for the prophylaxis and treatment of cosmetic or dermatological skin changes like those occurring for example during skin aging (such as, for example, dryness, roughness and formation of dryness wrinkles, itching, reduced refatting (e.g. after washing), visible dilations of vessels (telangiectasias, cuperosis), flaccidity and formation of creases and wrinkles, local hyperpigmentation, hypopigmentation and abnormal pigmentation (e.g. age spots), increased susceptibility to mechanical stress (e.g. chapping) and the like). They are also advantageously suitable for preventing the appearance of dry or rough skin.

The aqueous phase of the preparations of the invention may advantageously contain conventional cosmetic auxiliaries such as, for example, alcohols, especially those of low C number, preferably ethanol and/or isopropanol, diols or polyols of low C number, and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and, in particular, one or more thickeners which can advantageously be chosen from the group of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called carbopols, for example carbopols of types 980, 981, 1382, 2984, 5984, each singly or in combination. It is also possible and preferred to use moisturizers.

Substances or mixtures of substances referred to as moisturizers are those which confer on cosmetic or dermatological preparations the property of reducing the moisture loss from the stratum corneum (also called the transepidermal water loss (TEWL)) and/or have a beneficial effect on the hydration of the stratum corneum, after application or distribution on the surface of the skin.

Advantageous moisturizers for the purposes of the present invention are, for example, glycerol, lactic acid and/or lactates, especially sodium lactate, butylene glycol, propylene glycol, biosaccharides gum-1, glycine soya, ethylhexyloxyglycerin, pyrrolidonecarboxylic acid and urea. It is also particularly advantageous to use polymeric moisturizers from the group of polysaccharides which are soluble in water and/or swellable in water and/or gellable using water. Particularly advantageous are, for example, hyaluronic acid, chitosan and/or a fucose-rich polysaccharide which is listed in chemical abstracts under the registry number 178463-23-5 and is obtainable, for example, under the name Fucogel® 1000 from SOLABIA S.A.

The cosmetic or dermatological preparations of the invention may also advantageously, although not obligatorily, contain fillers which, for example, further improve the sensory and cosmetic properties of the formulations that induce or enhance for example a velvety or silky skin feel. Advantageous fillers for the purposes of the present invention are starch and starch derivatives (such as, for example, tapioca starch, distarch phosphate, aluminum- or sodium-starch octenylsuccinate and the like), pigments which have neither mainly a UV filter effect nor a coloring effect (such as, for example, boron nitride etc.) and/or Aerosils® (CAS No. 7631-86-9).

The oil phase of the formulations of the invention is advantageously chosen from the group of polar oils, for example from the group of lecithins and fatty acid triglycerides, namely the glycerol triesters of saturated and/or unsaturated, branched and/or unbranched alkanecarboxylic acids with a chain length of from 8 to 24, in particular 12 to 18, C atoms. The fatty acid triglycerides can, for example, advantageously be chosen from the group of synthetic, semisynthetic and natural oils such as, for example, coco glyceride, olive oil, sunflower oil, soybean oil, peanut oil, rapeseed oil, almond oil, palm oil, coconut oil, castor oil, wheatgerm oil, grapeseed oil, safflower oil, evening primrose oil, macadamia nut oil and more of the like.

Also advantageous according to the invention are, for example, natural waxes of animal and vegetable origin such as, for example, beeswax and other insect waxes, and berry wax, shea butter and/or lanolin (wool wax).

For the purposes of the present invention, further advantageous polar oil components can also be chosen from the group of esters of saturated and/or unsaturated, branched and/or unbranched alkanecarboxylic acids with a chain length of from 3 to 30 C atoms and saturated and/or unsaturated, branched and/or unbranched alcohols with a chain length of from 3 to 30 C atoms, and from the group of esters of aromatic carboxylic acids and saturated and/or unsaturated, branched and/or unbranched alcohols with a chain length of from 3 to 30 C atoms. Such ester oils can then be advantageously chosen from the group of octyl palmitate, octyl cocoate, octyl isostearate, octyldodeceyl myristate, octyidodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl iso-nonanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters such as, for example, jojoba oil.

The oil phase can advantageously also be chosen from the group of dialkyl ethers and dialkyl carbonates, advantageous examples being dicaprylyl ether (Cetiol OE) and/or dicaprylyl carbonate, for example the one obtainable under the proprietary name Cetiol CC from Cognis.

It is also preferred to choose the oil component(s) from the group of isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethylisosorbide. It is particularly advantageous if the oil phase in the formulations of the invention has a content of C₁₂₋₁₅-alkyl benzoate or consists entirely of the latter.

Further examples of advantageous oil components are butyloctyl salicylate (for example the one obtainable under the proprietary name Hallbrite B H B from C P Hall), hexadecyl benzoate and butyloctyl benzoate and mixtures thereof (Hallstar A B).

Any mixtures of such oil and wax components can also be advantageously employed for the purposes of the present invention.

The oil phase may likewise advantageously also contain nonpolar oils, for example those selected from the group of branched and unbranched hydrocarbons and waxes, in particular mineral oil, Vaseline (petrolatum), liquid paraffin, squalane and squalene, polyolefins, hydrogenated polyisobutenes and isohexadecane. The preferred substances among polyolefins are polydecenes.

The oil phase may also advantageously have a content of cyclic or linear silicone oils or consist completely of such oils, it being preferred, however, to use besides the silicone oil or silicone oils an additional content of other oil phase components.

Silicone oils are high molecular weight synthetic polymeric compounds in which silicon atoms are linked via oxygen atoms in the form of chains and/or networks, and the remaining valences of the silicon are saturated by hydrocarbon radicals (usually methyl groups and less commonly ethyl, propyl, phenyl groups inter alia). The systematic term for silicone oils is polyorganosiloxanes. The methyl-substituted polyorganosiloxanes which represent the most significant compounds in terms of quantity in this group and which have the following structural formula

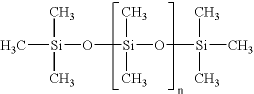

are also referred to as polydimethylsiloxane or dimethicone (INCI). Dimethicones differing in chain lengths and differing in molecular weights exist.

Particularly advantageous polyorganosiloxanes for the purposes of the present invention are, for example, dimethylpolysiloxanes [poly(dimethylsiloxane)], which are obtainable for example under the proprietary names Abil 10 to 10 000 from Th. Goldschmidt. Also advantageous are phenylmethylpolysiloxanes (INCI: phenyl dimethicone, phenyl trimethicone), cyclic silicones (octamethylcyclotetrasiloxane or decamethylcyclopentasiloxane) which are also referred to according to the INCI as cyclomethicones, amino-modified silicones (INCI: amodimethicone) and silicone waxes, e.g. polysiloxane-polyalkylene copolymers (INCI: stearyl dimethicone and cetyl dimethicone) and dialkoxydimethylpolysiloxanes (stearoxy dimethicone and behenoxy stearyl dimethicone) which are obtainable as various types of Abil wax from Th. Goldschmidt. However, other silicone oils can also be used advantageously for the purposes of the present invention, for example cetyldimethicone, hexamethylcyclotrisiloxane, polydimethylsiloxane, poly(methylphenylsiloxane).

It is also advantageous for the purposes of the present invention to produce cosmetic and dermatological preparations whose main purpose is not protection from sunlight but which, nevertheless, comprise a content of further UV-protective substances. Thus, for example, UV-A and UV-B filter substances are usually incorporated into day creams or makeup products. UV-protective substances as well as antioxidants and, if required, preservatives also represent effective protection of the preparations themselves against spoilage. Cosmetic and dermatological preparations which are in the form of a sunscreen composition are also favorable.

Accordingly, the preparations for the purposes of the present invention preferably contain at least one further UV-A, UV-B and/or broad-band filter substance. The formulations may, although not necessarily, optionally also contain one or more organic and/or inorganic pigment as UV filter substances, which may be present in the aqueous and/or the oil phase.

The preparations of the invention may also advantageously be in the form of so-called oil-free cosmetic or dermatological emulsions which comprise an aqueous phase and at least one UV filter substance which is liquid at room temperature and/or one or more silicone derivatives as further phase. Oil-free formulations for the purposes of the present invention may advantageously also comprise other lipophilic components—such as, for example, lipophilic active ingredients.

Particularly advantageous UV filter substances which are liquid at room temperature for the purposes of the present invention are homomenthyl salicylate (INCI: homosalate), 2-ethylhexyl 2-cyano-3,3-diphenylacrylate (INCI: octocrylene), 2-ethylhexyl 2-hydroxybenzoate (2-ethylhexyl salicylate, octyl salicylate, INCI: octyl salicylate) and esters of cinnamic acid, preferably 4-methoxycinnamic acid (2-ethylhexyl) ester (2-ethylhexyl 4-methoxycinnamate, INCI: octyl methoxycinnamate) and 4-methoxycinnamic acid isopentyl ester (isopentyl 4-methoxycinnamate, INCI: isoamyl p-methoxycinnamate).

Preferred inorganic pigments are metal oxides and/or other metal compounds which are slightly soluble or insoluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and the sulfate of barium (BeSO₄).

The pigments can advantageously for the purposes of the present invention also be used in the form of commercially available oily or aqueous predispersions. It is possible and advantageous to add dispersion aids and/or solubilizers to these predispersions.

The pigments may advantageously according to the invention be surface-treated (“coated”), the intention being for example to form or retain a hydrophilic, amphiphilic or hydrophobic character. This surface treatment may consist of providing the pigments by processes known per se with a thin hydrophilic and/or hydrophobic inorganic and/or organic layer. The various surface coatings may for the purposes of the present invention also contain water.

Inorganic surface coatings for the purposes of the present invention may consist of aluminum oxide (Al₂O₃), aluminum hydroxide Al(OH)₃ or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may occur alone, in combination and/or in combination with organic coating materials.

Organic surface coatings for the purposes of the present invention may consist of vegetable or animal aluminum stearate, vegetable or animal stearic acid, lauric acid, dimethylpolysiloxane (also: dimethicones), methylpolysiloxane (methicones), simethicones (a mixture of dimethylpolysiloxane with an average chain length of from 200 to 350 dimethylsiloxane units and silica gel) or alginic acid. These organic surface coatings may occur alone, in combination and/or in combination with inorganic coating materials.

Zinc oxide particles and predispersions of zinc oxide particles which are suitable according to the invention are obtainable under the following proprietary names from the stated companies:

| Proprietary name | Coating | Manufacturer |
|---|---|---|
| Z-Cote HP1 | 2% dimethicone | BASF |
| Z-Cote | / | BASF |
| ZnO NDM | 5% dimethicone | H&R |

Suitable titanium dioxide particles and predispersions of titanium dioxide particles are obtainable under the following proprietary names from the stated companies:

| Proprietary name | Coating | Manufacturer |
|---|---|---|
| MT-100TV | Aluminum hydroxide/stearic | Tayca Corporation |
| | acid | |
| MT-100Z | Aluminum hydroxide/stearic | Tayca Corporation |
| | acid | |
| Eusolex T-2000 | Alumina/simethicone | Merck KgaA |
| Titandioxid T805 | Octyltrimethylsilane | Degussa |
| (Uvinul TiO₂) | | |
| Tioveil AQ 10PG | Alumina/silica | Solaveil/ |
| | | Uniquema |

Further advantageous pigments are latex particles. Latex particles which are advantageous according to the invention are described in the following publications: U.S. Pat. No. 5,663,213 and EP 0 761 201. Particularly advantageous latex particles are those formed from water and styrene/acrylate copolymers and available for example under the proprietary name “Alliance SunSphere” from Rohm & Haas.

Advantageous UV filter substances for the purposes of the present invention are also so-called broad-band filters, i.e. filter substances which absorb both UV-A and UV-B rays.

Advantageous broad-band filters or UV-B filter substances are, for example, triazine derivatives such as, for example,

2,4-bis-{[4-(2-ethylhexyloxy)-2-hydroxy]phenyl}-6-(4-methoxyphenyl)-1,3,5-triazine (INCI: methylene bis-benzotriazolmethylbutylphenol), which is obtainable under the proprietary name Tinosorb® S from CIBA-Chemikalien GmbH;

dioctylbutylamidotriazone (INCI: diethylhexylbutamidotriazone), which is obtainable under the proprietary name UVASORB HEB from Sigma 3V;

4,4′,4″-(1,3,5-triazine-2,4,6-triyltriimino)trisbenzoic acid tris(2-ethylhexyl ester), also: 2,4,6-tris[anilino(p-carbo-2′-ethyl-1′-hexyloxy)]-1,3,5-triazine (INCI: octyl triazone), 5 which is marketed by BASF Aktiengesellschaft under the trademark UVINUL® T 150.

An advantageous broad-band filter for the purposes of the present invention is also 2,2′-methylenebis(6-(2H-benzotriazol-2-yl)-4-(1,1,3,3-tetramethylbutyl)phenol) (INCI: bisoctyltriazol), which is obtainable under the proprietary name Tinosorbe® M from CIBA-Chemikalien GmbH.

An advantageous broad-band filter for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenyl (CAS No.: 155633-54-8) with the INCI name drometrizole trisiloxane.

The other UV filter substances may be oil-soluble. Advantageous oil-soluble filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

derivatives of benzophenone, preferably 2-hydroxy-4-methoxybenzophenone, 2-hydroxy-4-methoxy-4′-methyl benzophenone, 2,2′-dihydroxy-4-methoxy-benzophenone

and UV filters bound to polymers.

A further light-protective filter substance which can advantageously be used according to the invention is ethylhexyl 2-cyano-3,3-diphenylacrylate (octocrylene) which is obtainable from BASF under the name Uvinul® N 539.

Particularly advantageous preparations for the purposes of the present invention are those which display high or very high UV-A protection, contain, besides the filter substance(s) of the invention, preferably also further UV-A and/or broad-band filters, especially dibenzoylmethane derivatives [for example 4-(tert-butyl)-4′-methoxydibenzoylmethane] and/or 2,4-bis{[4-(2-ethylhexyloxy)-2-hydroxy]phenyl}-6-(4-methoxyphenyl)-1,3,5-triazine, each singly or in any combinations with one another.

The list of UV filters mentioned which can be employed for the purposes of the present invention is, of course, not intended to be limiting.

The preparations of the invention advantageously contain the substances which absorb UV rays in the UV-A and/or UV-B ranges in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair and skin from the entire range of ultraviolet radiation.

It may also be advantageous where appropriate to incorporate film formers into the cosmetic or dermatological preparations of the invention, for example in order to improve the water resistance of the preparations or to increase the effectiveness of UV protection (UV-A and/or UV-B boosting). Both water-soluble or dispersible and fat-soluble film formers are suitable, each singly or in combination with one another.

Advantageous water-soluble or dispersible film formers are, for example, polyurethanes (e.g. the Avalure® types from Goodrich), dimethicone copolyol polyacrylates (Silsoft Surface® from Witco Organo Silicones Group), PVPNA (VA=vinyl acetate) copolymer (Luviscol VA 64 powder from BASF) etc.

Advantageous fat-soluble film formers are, for example, the film formers from the group of polymers based on polyvinylpyrrolidone (PVP)

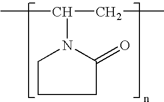

Copolymers of polyvinylpyrrolidone are particularly preferred, for example the PVP hexadecene copolymer and the PVP eicosene copolymer, which are obtainable under the proprietary names Antaron V216 and Antaron V220 from GAF Chemicals Cooperation, and Tricontayl PVP and more of the like.

The following examples are intended to illustrate the present invention without restricting it. The numerical values in the examples mean percentages by weight based on the total weight of the respective preparations.

### EXAMPLES

1. O/W sunscreen emulsions

| | 1 | 2 | 3 | 4 | 5 | 6 | 7 |
|---|---|---|---|---|---|---|---|
| Glyceryl monostearate SE | 0.50 | 1.00 | 3.00 | | | 1.50 | |
| Glyceryl stearate citrate | 2.00 | | | 1.00 | 2.00 | | 2.50 |
| Stearic acid | | 3.00 | | 2.00 | | | |
| PEG-40 stearate | 0.50 | | | | | 2.00 | |
| Cetyl phosphate | | | | | 1.00 | | |
| Stearyl alcohol | | | 3.00 | | | 2.00 | 0.50 |
| Cetyl alcohol | 2.50 | 1.00 | | 1.50 | 0.50 | | 2.00 |
| Ethylhexyl methoxycinnamate | | | | 5.00 | 6.00 | | 8.00 |
| Bis-ethylhexyloxyphenol | | 1.50 | | 2.00 | 2.50 | | 2.50 |
| methoxyphenyl triazine | | | | | | | |
| Butyl methoxydibenzoylmethane | 1.00 | 3.00 | 2.00 | 1.50 | 2.80 | 2.00 | 1.50 |
| Disodium phenyl | 2.50 | 0.50 | 0.50 | 2.00 | 1.00 | 1.70 | 0.30 |
| dibenzimidazole | | | | | | | |
| tetrasulfonate | | | | | | | |
| Ethylhexyl triazone | 4.00 | | 3.00 | 4.00 | 4.00 | 2.00 | |
| 4-Methylbenzylidene | 4.00 | 4.00 | | | 2.00 | 4.00 | 2.00 |
| camphor | | | | | | | |
| Octocrylene | | 4.00 | | | | | 2.50 |
| Diethylhexyl butamido | 1.00 | | | 2.00 | 1.00 | | |
| triazone | | | | | | | |
| Phenylbenzmidazole sulfonic | 0.50 | | | 3.00 | | | |
| acid | | | | | | | |
| Methylene bis-benzotriazolyl | 2.00 | | 0.50 | 1.50 | 2.50 | | |
| tetramethylbutylphenol | | | | | | | |
| Benzophenone-3 | | | | 5.50 | | | |
| Isoamyl p-methoxycinnamate | | 1.50 | | | | | |
| Homosalate | | 2.00 | | | | | |
| Ethylhexyl salicylate | | | 3.00 | | | | 5.00 |
| Drometrizole trisiloxane | | | 0.50 | | | 1.00 | |
| Terephthalidene dicamphor | | 1.50 | | | 1.00 | 0.50 | |
| sulfonic acid | | | | | | | |
| Diethylhexyl 2,6-naphthalate | 3.50 | 4.80 | 7.00 | 9.50 | 6.70 | 5.50 | 8.00 |
| Titanium dioxide MT-100Z | 1.00 | 1.50 | | 3.00 | 2.00 | 2.00 | |
| Zinc oxide HP1 | | | 1.50 | 1.00 | | 2.00 | 3.00 |
| C12-15 alkyl benzoate | | 2.50 | | | 4.00 | 7.00 | 5.00 |
| Dicaprylyl ether | | | 3.50 | | 2.00 | | |
| Butylene glycol dicaprylate/ | 5.00 | | | 6.00 | | | |
| dicaprate | | | | | | | |
| Dicaprylyl carbonate | | | 6.00 | | | 2.00 | 2.00 |
| Dimethicone | | 0.50 | 1.00 | | 2.00 | | |
| Cyclomethicone | 2.00 | | | 0.50 | | | 0.50 |
| Shea butter | | 2.00 | | | | | 0.50 |
| PVP hexadecene copolymer | 0.50 | | | 0.50 | 1.00 | | 1.00 |
| Tricontanyl PVP | | 0.50 | 1.00 | | | | 1.00 |
| Glycerol | 3.00 | 7.50 | | 7.50 | 5.00 | | 2.50 |
| Xanthan gum | 0.15 | | 0.05 | | | | 0.30 |
| Sodium carbomer | | 0.20 | 0.10 | 0.20 | | | |
| Vitamin E acetate | 0.50 | | 0.25 | | 0.75 | | 1.00 |
| Polyurethane | | 0.50 | | 0.50 | | 1.00 | |
| Styrene/acrylate copolymer | 0.80 | | 3.00 | 1.50 | | | |
| DMDM hydantoin | | 0.60 | 0.40 | 0.20 | | | |
| Konkaben LMB ® | | | | 0.18 | 0.20 | 0.10 | 0.15 |
| Methylparaben | 0.15 | | 0.25 | | 0.50 | | |
| Phenoxyethanol | 1.00 | 0.40 | | 0.40 | 0.50 | 0.40 | 0.60 |
| Ethanol | | 2.00 | 1.50 | | 3.00 | | 1.00 |
| Perfume | 0.20 | 0.20 | 0.20 | 0.20 | 0.20 | 0.20 | 0.20 |
| Water | ad | ad | ad | ad | ad | ad 100 | ad 100 |
| | 100 | 100 | 100 | 100 | 100 | | |

| | | | | |
|---|---|---|---|---|
| 1 | 2 | 3 | 4 | 5 |

2. Hydrodispersions

| | | | | | |
|---|---|---|---|---|---|
| Ceteareth-20 | 1.00 | | | 0.5 | |
| Cetyl alcohol | | | 1.00 | | |
| Sodium carbomer | | 0.20 | | 0.30 | |
| Acrylates/C10-30 alkyl acrylate | 0.50 | | 0.40 | 0.10 | 0.10 |
| crosspolymer | | | | | |
| Xanthan gum | | 0.30 | 0.15 | | 0.50 |
| Ethylhexyl methoxycinnamate | | | | 5.00 | 8.00 |
| Bis-Ethylhexyloxyphenol | | 1.50 | | 2.00 | 2.50 |
| methoxyphenyl triazine | | | | | |
| Butyl | 1.00 | 0.50 | 2.00 | 3.00 | 2.50 |
| methoxydibenzoylmethane | | | | | |
| Disodium phenyl | 0.50 | 1.80 | 1.50 | 2.00 | 3.00 |
| dibenzimidazole tetrasulfonate | | | | | |
| Ethylhexyl triazone | 4.00 | | 3.00 | 4.00 | |
| 4-Methylbenzylidene camphor | 4.00 | | | | 2.00 |
| Octocrylene | | 4.00 | 3.90 | | 2.50 |
| Diethylhexyl butamido triazone | 1.00 | | | 2.00 | |
| Phenylbenzmidazole sulfonic | 0.50 | | | 3.00 | |
| acid | | | | | |
| Methylene bis-benzotriazolyl | 2.50 | 0.50 | | | 0.80 |
| tetramethylbuthylphenol | | | | | |
| Drometrizole trisiloxane | | | 1.00 | | 1.50 |
| Terephthalidene dicamphor | | 0.50 | | | 1.00 |
| sulfonic acid | | | | | |
| Diethylhexyl 2,6-naphthalate | 4.50 | 8.00 | 7.20 | 5.50 | 9.80 |
| Titanium dioxide MT-100TV | 0.50 | | 2.00 | | 1.00 |
| Zinc oxide HP1 | | | 1.00 | 2.00 | 3.00 |
| C12-15 alkyl benzoate | 2.00 | 2.50 | | | |
| Dicaprylyl ether | | 4.00 | | | |
| Butylene glycol dicaprylate/ | 4.00 | | 2.00 | 6.00 | |
| dicaprate | | | | | |
| Dicaprylyl carbonate | | 2.00 | 6.00 | | |
| Dimethicone | | 0.50 | 1.00 | | |
| Phenyltrimethicone | 2.00 | | | 0.50 | 2.00 |
| Shea butter | | 2.00 | | | |
| PVP hexadecene copolymer | 0.50 | | | 0.50 | 1.00 |
| Tricontanyl PVP | 0.50 | | 1.00 | | |
| Ethylhexylglycerol | | | 1.00 | | 0.50 |
| Glycerol | 3.00 | 7.50 | | 7.50 | 2.50 |
| Glycine soya | | | 1.50 | | |
| Vitamin E acetate | 0.50 | | 0.25 | | 1.00 |
| Polyurethane | | 0.60 | 1.50 | 1.00 | |
| Styrene/acrylate copolymer | | 2.50 | 0.50 | | 2.00 |
| DMDM hydantoin | | 0.60 | 0.40 | 0.20 | |
| Konkaben LMB ® | 0.20 | | | | 0.15 |
| Methylparaben | 0.50 | | 0.25 | 0.15 | |
| Phenoxyethanol | 0.50 | 0.40 | | 1.00 | 0.60 |
| Ethanol | 3.00 | 2.00 | 1.50 | | 1.00 |
| Perfume | 0.20 | 0.20 | 0.20 | 0.20 | 0.20 |
| Water | ad 100 | ad 100 | ad 100 | ad 100 | ad 100 |

3. W/O sunscreen emulsions

| | | | | | |
|---|---|---|---|---|---|
| Cetyldimethicone copolyol | | 2.50 | | 4.00 | |
| Polyglyceryl 2-dipolyhydroxystearate | 5.00 | | | | 4.50 |
| PEG 30 dipolyhydroxystearate | | | 5.00 | | |
| Ethylhexyl methoxycinnamate | | 8.00 | | 5.00 | 4.00 |
| Bis-Ethylhexyloxyphenol | 2.00 | 2.50 | | 2.00 | 2.50 |
| methoxyphenyl triazone | | | | | |
| Butyl | 0.50 | 3.00 | 2.00 | 1.00 | 0.70 |
| methoxydibenzoylmethane | | | | | |
| Disodium phenyl | 0.50 | 1.00 | 1.60 | 2.00 | 2.60 |
| dibenzimidazole tetrasulfonate | | | | | |
| Ethylhexyl triazone | | | 3.00 | 4.00 | |
| 4-Methylbenzylidene camphor | | 2.00 | | 4.00 | 2.00 |
| Octocrylene | 0.90 | 2.50 | 3.90 | | 2.50 |
| Diethylhexyl butamido triazone | 1.00 | | | 2.00 | |
| Phenylbenzmidazole sulfonic | 0.50 | | | 3.00 | 2.00 |
| acid | | | | | |
| Methylene bis-benzotriazolyl | | | 2.00 | 0.50 | |
| tetramethylbutylphenol | | | | | |
| Drometrizole trisiloxane | | 1.00 | | | 1.50 |
| Terephthalidene dicamphor | | | 1.00 | | 0.50 |
| sulfonic acid | | | | | |
| Diethylhexyl 2,6-naphthalate | 7.50 | 5.50 | 3.50 | 8.80 | 9.70 |
| Titanium dioxide T805 | | 2.00 | 1.50 | | 3.00 |
| Zinc oxide NDM | 1.00 | | 3.00 | | 2.00 |
| Mineral oil | | | 10.0 | | 8.00 |
| C12-15 alkyl benzoate | | | | 9.00 | |
| Dicaprylyl ether | 10.00 | | | | 7.00 |
| Butylene glycol dicaprylate/ | | | 2.00 | 8.00 | 4.00 |
| dicaprate | | | | | |
| Dicaprylyl carbonate | 5.00 | | 6.00 | | |
| Dimethicone | | 4.00 | 1.00 | 5.00 | |
| Cyclomethicone | 2.00 | 25.00 | | | 2.00 |
| Shea butter | | | 3.00 | | |
| PVP hexadecene copolymer | 0.50 | | | 0.50 | 1.00 |
| Tricontanyl PVP | | | 0.50 | 1.00 | 0.50 |
| Ethylhexylglycerol | | 0.30 | 1.00 | | 0.50 |
| Glycerol | 3.00 | 7.50 | | 7.50 | 2.50 |
| Glycine soya | | 1.00 | 1.50 | | |
| MgSO₄ | 1.00 | 0.50 | | 0.50 | |
| MgCl₂ | | | 1.00 | | 0.70 |
| Vitamin E acetate | 0.50 | | 0.25 | | 1.00 |
| Styrene/acrylate copolymer | 0.50 | | | 2.50 | |
| Polyurethane | | 0.50 | | 1.50 | |
| DMDM hydantoin | | 0.60 | 0.40 | 0.20 | |
| Methylparaben | 0.50 | | 0.25 | 0.15 | |
| Phenoxyethanol | 0.50 | 0.40 | | 1.00 | 0.60 |
| Ethanol | 3.00 | | 1.50 | | 1.00 |
| Perfume | 0.20 | 0.20 | 0.20 | 0.20 | 0.20 |
| Water | ad 100 | ad 100 | ad 100 | ad 100 | ad 100 |

4. Solid-stabilized emulsions

| | 1 | 2 | 3 | 4 | 5 |
|---|---|---|---|---|---|
| Mineral oil | | | 16.0 | 16.0 | |
| Octyldodecanol | 9.0 | 9.0 | 5.0 | | |
| Caprylic/capric triglyceride | 9.0 | 9.0 | 6.0 | | |
| C12-15 alkyl benzoate | | | | 5.0 | 8.0 |
| Butylene glycol dicaprylate/ | | | | | 8.0 |
| dicaprate | | | | | |
| Dicaprylyl ether | 9.0 | | | 4.0 | |
| Dicaprylyl carbonate | | 9.0 | | | |
| Hydroxyoctacosanyl | 2.0 | 2.0 | 2.0 | 2.0 | 1.5 |
| hydroxystearate | | | | | |
| Disteardimonium hectorite | 1.0 | 0.75 | 0.5 | 0.5 | 0.25 |
| Microcrystalline wax + liquid | | | | | 5.0 |
| paraffin | | | | | |
| Hydroxypropyl methylcellulose | | | | | 0.05 |
| Dimethicone | | | | | 3.0 |
| Butyl | 2.00 | 0.50 | 3.50 | 1.50 | 0.50 |
| methoxydibenzoylmethane | | | | | |
| Ethylhexyl methoxycinnamate | | | | | 3.0 |
| 4-methylbenzylidene camphor | | | | | 4.0 |
| Diethylhexyl butamido triazone | | | | | 4.0 |
| Methylene bis-benzotriazolyl | 0.50 | | | 2.0 | |
| tetramethylbutylphenol | | | | | |
| Drometrizole trisiloxane | | 0.50 | | 1.00 | |
| Terephthalidene dicamphor | | 1.00 | 0.50 | | 1.50 |
| sulfonic acid | | | | | |
| Disodium phenyl | 2.50 | 2.00 | 3.10 | 1.50 | 0.50 |
| dibenzimidazole tetrasulfonate | | | | | |
| Titanium dioxide + alumina + | | 2.0 | 4.0 | 2.0 | 4.0 |
| simethicone + water | | | | | |
| Titanium dioxide + | | | | | 3.0 |
| trimethoxycaprylylsilane | | | | | |
| Zinc oxide HP1 | | | | 6.0 | |
| Silica dimethyl silylate | | | 1.0 | | |
| Boron nitride | 2.0 | | | | |
| Starch/sodium metaphosphate | | 0.5 | | | |
| polymer | | | | | |
| Diethylhexyl 2,6-naphthalate | 5.00 | 7.00 | 8.50 | 3.00 | 4.50 |
| Tapioca starch | | | | 1.0 | |
| Polyurethane | 0.20 | | 1.50 | 0.50 | |
| Styrene/acrylate copolymer | | 2.00 | | | 3.00 |
| Sodium chloride | 1.0 | 1.0 | 1.0 | 1.0 | 1.0 |
| Glycerol | 5.0 | 10.0 | 3.0 | 6.0 | 10.0 |
| Trisodium EDTA | | 1.0 | | 1.0 | |
| Methylparaben | 0.21 | | | | 0.2 |
| Propylparaben | 0.07 | | | | |
| Phenoxyethanol | 0.5 | | 0.4 | 0.4 | 0.5 |
| Hexamidine diisethionate | | | | | 0.08 |
| Diazolidinyl urea | | | 0.28 | 0.28 | |
| Alcohol | | | | 2.5 | |
| Perfume | q.s. | q.s. | q.s. | q.s. | q.s. |
| Water | ad 100 | ad 100 | ad 100 | ad 100 | ad 100 |
| | 6 | 7 | 8 | 9 | 10 |
| Mineral oil | | | | | 16.0 |
| Octyldodecanol | 6.0 | | 7.5 | 7.5 | 5.0 |
| Caprylic/capric triglyceride | | | | | 6.0 |
| C12-15 alkyl benzoate | 7.0 | 8.0 | 7.5 | 7.5 | |
| Butylene glycol dicaprylate/ | 4.0 | 8.0 | | | |
| dicaprate | | | | | |
| Dicaprylyl ether | | 8.0 | 7.5 | 7.5 | |
| Dicaprylyl carbonate | 4.0 | | | | |
| Hydroxyoctacosanyl | 2.0 | 2.0 | 2.0 | 2.0 | 1.5 |
| hydroxystearate | | | | | |
| PVP/hexadecene copolymer | | | | 1.0 | 0.7 |
| Disteardimonium hectorite | 1.0 | 1.0 | 1.0 | 0.5 | 1.0 |
| Dimethicone | | 2.0 | | | |
| Cyclomethicone | | | | 2.0 | |
| Ethylhexyl methoxycinnamate | 5.0 | | 5.0 | | |
| Butyl | 3.00 | 2.0 | 0.50 | 1.80 | 1.0 |
| methoxydibenzoylmethane | | | | | |
| 4-Methylbenzylidene camphor | | 4.0 | | | 2.0 |
| Ethylhexyltriazone | 2.0 | 2.0 | | | 1.0 |
| Methylene bis-benzotriazolyl | | 3.00 | | 2.50 | |
| tetramethylbuthylphenol | | | | | |
| Bis-Ethylhexyloxyphenol | 2.5 | | 2.5 | | |
| methoxyphenyl triazine | | | | | |
| Titanium dioxide + alumina + | 1.5 | 2.0 | 4.0 | 0.5 | 1.5 |
| simethicone + water | | | | | |
| Titanium dioxide + | | | 2.0 | | |
| trimethoxycaprylylsilane | | | | | |
| Zinc oxide | | | 2.0 | | |
| Phenylbenzimidazole sulfonic | 2.0 | | | | |
| acid | | | | | |
| Disodium phenyl | 2.50 | 1.00 | 0.60 | 1.50 | 3.00 |
| dibenzimidazole tetrasulfonate | | | | | |
| Boron nitride | | | | | 0.5 |
| Starch/sodium metaphosphate | 0.5 | | 1.5 | | |
| polymer | | | | | |
| Corn starch modified | | 1.0 | | | |
| Acrylate copolymer | | | | 0.25 | |
| Talc | | | | 2.0 | |
| Sodium chloride | 1.0 | 1.0 | 1.0 | | |
| Diethylhexyl 2,6-naphthalate | 4.00 | 6.50 | 7.50 | 9.50 | 5.00 |
| Polyurethane | | 0.50 | 1.50 | | 0.40 |
| Styrene/acrylate copolymer | 1.50 | | | | 3.00 |
| Magnesium sulfate | | | | | 0.70 |
| 45% sodium hydroxide solution | 0.5 | 0.5 | | | |
| Glycerol | 5.0 | 7.5 | 5.0 | 10.0 | 3.0 |
| Trisodium EDTA | | 1.0 | 1.0 | | 1.0 |
| Propylene carbonate | 0.33 | 0.33 | 0.33 | | 0.33 |
| Methylparaben | 0.21 | 0.21 | 0.2 | 0.2 | 0.21 |
| Propylparaben | 0.07 | 0.07 | | | 0.07 |
| Phenoxyethanol | 0.5 | 0.5 | 0.5 | 0.5 | 0.5 |
| Hexamidine diisethionate | | | 0.08 | 0.08 | |
| Alcohol | | 5.0 | | | |
| Perfume | q.s. | q.s. | q.s. | q.s. | q.s. |
| Water | ad 100 | ad 100 | ad 100 | ad 100 | ad 100 |

That which is claimed:

1. A cosmetic or dermatological formulation comprising:
(a) at least one water-soluble UV filter substance, and
(b) at least one dialkyl naphthalate having the structural formula 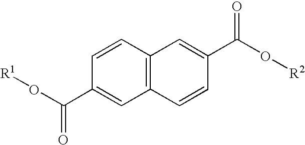
wherein R¹ and R² are selected independently of one another from the group consisting of branched and unbranched alkyl groups having 6 to 24 carbon atoms.

2. The formulation as claimed in claim 1, wherein at least one of R¹ and R² is a branched alkyl group having 6 to 10 carbon atoms.

3. The formulation as claimed in claim 1, wherein R¹ and R² are branched alkyl groups having 6 to 10 carbon atoms.

4. The formulation as claimed in claim 1, wherein the at least one dialkyl naphthalate comprises diethylhexyl naphthalate.

5. The formulation as claimed in claim 1, wherein the at least one dialkyl naphthalate is present in an amount of 0.001 to 30 weight % based on the total weight of the formulation.

6. The formulation as claimed in claim 5, wherein the at least one dialkyl naphthalate is present in an amount of 0.01 to 20 weight % based on the total weight of the formulation.

7. The formulation as claimed in claim 5, wherein the at least one dialkyl naphthalate is present in an amount of 0.5 to 15 weight % based on the total weight of the formulation.

8. The formulation as claimed in claim 1, wherein the at least one water-soluble UV filter substance is a sulfonated UV filter.

9. The formulation as claimed in claim 8, wherein the sulfonated UV filter is selected from the group consisting of phenylene-1,4-bis(2-benzimidazyl)-3,3′-5,5′-tetrasulfonic acid and its corresponding salts, 2-phenylbenzimidazole-5-sulfonic acid and its corresponding salts, and mixtures thereof.

10. The formulation as claimed in claim 9, wherein the sulfonated UV filter is phenylene-1,4-bis(2-benzimidazyl)-3,3′-5,5′-tetrasulfonic acid bissodium salt.

11. The formulation as claimed in claim 1, wherein the at least one water-soluble UV filter substance comprises sulfonic acid derivatives of 3-benzylidenecamphor and salts thereof.

12. The formulation as claimed in claim 1, wherein the at least one water-soluble UV filter substance is present in an amount of 0.01 to 20 weight % based on the total weight of the formulation.

13. The formulation as claimed in claim 12, wherein the at least one water-soluble UV filter substance is present in an amount of 0.1 to 10 weight % based on the total weight of the formulation.

14. The formulation as claimed in claim 1, further comprising a substance selected from the group consisting of flavone glycosides, vitamin E, vitamin E derivatives, and mixtures thereof.

15. The formulation as claimed in claim 14, wherein the flavone glycoside is α-glucosylrutin.

16. The formulation as claimed in claim 1, further comprising at least one UV filter substance selected from the group consisting of UV-A filters, UV-B filters, broad-band filters, triazines, benzotriazoles, organic pigments, inorganic pigments, and mixtures thereof.

17. The formulation as claimed in claim 16, wherein the at least one UV filter substance includes at least one dibenzoylmethane derivative.

18. The formulation as claimed in claim 17, wherein the dibenzoylmethane derivative is selected from the group consisting of 4-(tert-butyl)-4′-methoxydibenzoylmethane, 2,4-bis{[4-(2-ethylhexyloxy)-2-hydroxy]phenyl}-6-(4-methoxyphenyl)-1,3,5-triazine, and mixtures thereof.

19. The formulation as claimed in claim 16, wherein the UV-A and UV-B filters are present in an amount of 0.1 to 30 weight % based upon the total weight of the formulation.

20. The formulation as claimed in claim 19, wherein the UV-A and UV-B filters are present in an amount of 0.5 to 20 weight % based upon the total weight of the formulation.

21. The formulation as claimed in claim 19, wherein the UV-A and UV-B filters are present in an amount of 1 to 15 weight % based upon the total weight of the formulation.

22. A method for moisturizing skin comprising applying to the skin a cosmetic or dermatological formulation comprising
(a) at least one water-soluble UV filter substance, and
(b) at least one dialkyl naphthalate having the structural formula 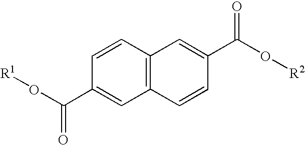
wherein R¹ and R² are selected independently of one another from the group consisting of branched and unbranched alkyl groups having 6 to 24 carbon atoms.

23. A method for protecting skin from light-induced skin aging comprising applying to the skin a cosmetic or dermatological formulation comprising:
(a) at least one water-soluble UV filter substance, and
(b) at least one dialkyl naphthalate having the structural formula 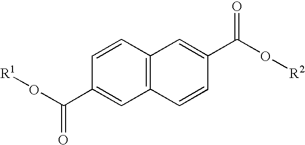
wherein R¹ and R² are selected independently of one another from the group consisting of branched and unbranched alkyl groups having 6 to 24 carbon atoms.

24. A method for stabilizing dibenzoylmethane derivatives against decomposition induced by UV radiation in cosmetic or dermatological formulations comprising at least one water-soluble UV filter substance, said method comprising adding to the cosmetic or dermatological formulation at least one dialkyl naphthalate having the structural formula 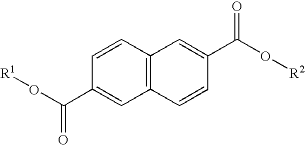
wherein R¹ and R² are selected independently of one another from the group consisting of branched and unbranched alkyl groups having 6 to 24 carbon atoms.
